(19) United States
(12) Patent Application Publication
(10) Pub. No.: US 2011/0206611 A1
(43) Pub. Date: Aug. 25, 2011

(54) DNA Dendrimers as Thermal Ablation Devices

(75) Inventors: James Kadushin, Gilbertsville, PA (US); Robert C. Getts, Collegeville, PA (US)

(73) Assignee: Genisphere, LLC, Hatfield, PA (US)

(21) Appl. No.: 13/033,074

(22) Filed: Feb. 23, 2011

Related U.S. Application Data
(60) Provisional application No. 61/307,622, filed on Feb. 24, 2010.

(51) Int. Cl. A61K 49/00; C07H 21/04; C07K 14/00; C07K 2/00; C07K 16/00; C12N 13/00; A61K 31/711; C07H 1/00; C12Q 1/68; A61M 37/00; B82Y 5/00

(52) U.S. Cl. 424/9.1; 536/23.1; 530/409; 530/300; 530/391.1; 435/173.1; 514/44 R; 536/25.3; 435/6.1; 604/20; 977/911; 977/927

(57) ABSTRACT

DNA dendrimers for targeted delivery of radiation absorbing nanoparticles and thermal ablation of cells and tissues are provided. Also provided are methods of making and methods of using the DNA dendrimers.

### CROSS REFERENCE TO RELATED APPLICATIONS

This application claims the benefit of U.S. Provisional Application Ser. No. 61/307,622, filed Feb. 24, 2010.

### TECHNICAL FIELD

The invention relates to materials and methods for thermal ablation of cells and tissues using targeted delivery of radiation absorbing nanoparticles.

### BACKGROUND

The 3DNA dendrimer is a proprietary dendritic molecule comprised solely of DNA. As a class, dendrimers are complex, highly branched molecules built from interconnected natural or synthetic monomeric subunits. A 3DNA® dendrimer is constructed from DNA monomers, each of which is made from two DNA strands that share a region of sequence complementarity located in the central portion of each strand (FIG. 1). Monomers are combined during the manufacturing process to prepare DNA dendrimers of different sizes and shapes (FIG. 2). In order to prevent DNA dendrimers from falling apart over time, chemical “spot welds” are added to the growing assembly during the process using UV light via the intercalation and activation of psoralen cross-linkers. Dendrimers are purified according to their size and molecular weight on denaturing sucrose gradients after ultracentrifugation (FIG. 3).

DNA dendrimers have the ability to be covalently and non-covalently bound to a large variety of different types of molecules and particles. These molecules and particles have typically been used as signaling and targeting devices on DNA dendrimers, allowing the targeting of DNA dendrimers to specific molecular targets and the detection of the binding of the dendrimers to the targets via the detection of the signaling moieties. Signal generating moieties have included a large number of fluorescent dyes, haptens, enzymes and other molecular materials, as well as particles such as gold nanoparticles and quantum dots. Targeting devices include DNA, RNA and PNA oligonucleotides, antibodies, antibody fragments, haptens, aptamers, peptides and others. These DNA dendrimer constructs have been used as signal amplifiers in a large variety of in-vitro applications, generally for the detection of specific nucleic acids and proteins, but also as detection devices in electronic devices. Applications include signal amplification on DNA and protein microarrays, ELISAs and ELOSAs, Luminex bead assays, in-situ hybridization, and others. The use of labeled and targeted DNA dendrimers has been extensively published in research studies and these materials are available as commercial research products sold or produced by Genisphere LLC (Hatfield, Pa.).

DNA dendrimers have also been shown to have potential use as delivery and transfection devices in both in-vitro and in-vivo applications. See, e.g., U.S. 2005/0089890, WO 2008/147526 and WO 2010/017544, each of which is incorporated by reference in its entirety. Specifically, DNA dendrimers are bound with targeting devices (e.g. an antibody specific for a cell surface feature capable of eliciting an cellular endocytotic internalization event) which bind to surface features on cells targeted to receive the delivery of a cargo (e.g. a drug). Cargos may be passively associated with the targeted DNA dendrimer and enter the cell simply by spatial association with the dendrimer, or cargos may be directly bound to the dendrimer via a number of attachment strategies.

Gold nanoparticles (and nanoparticles containing other metals including silver, cadmium, iron and others) subjected to RF fields of between 100 and 2000 watts (at a wavelength of 13.56 MHz) for up to 5 minutes have been used for thermal ablation of cells in both in-vitro and in-vivo applications. See, e.g., Cardinal et al., 2008, Surgery 144:125-132 and Gannon et al., 2008, J. Nanobiotechnology 6:2, each of which is incorporated by reference in its entirety. Such methods may be referred to as radiofrequency ablation (RFA), and have been used in clinical practice to treat tumors. However, there is a particular need to develop improved thermal ablation technologies for treatment of tumors, as current treatments are invasive procedures that require insertion of needle electrodes directly into the tumor, complete tumor destruction is difficult to achieve particularly for larger tumors and the treatment is relatively non-specific with both malignant and normal tissues around the electrode being subjected to thermal injury. Penetration of human tissue by focused external RF energy fields is effective, but use of an external energy source requires the presence of an intracellular or intratumoral agent that responds specifically to RFA to target thermal therapy to malignant cells. In addition, there is a need for compositions and methods that maximize the thermal ablation capability delivered by a targeting or carrier molecule, thus minimizing the amount of the thermal ablation composition that must be delivered to the patient and reducing any potential toxicity of the composition itself. Further, to deliver nanoparticles to a targeted tissue a carrier must be large enough to avoid clearance by the reticuloendothelial system (RES) but small enough to enter tumor tissue from the circulation (e.g., by extravasation). This places size constraints on such compositions.

### SUMMARY

The present invention includes the use of DNA dendrimers containing particles capable of being heated in-situ via the use of remote electromagnetic fields, such as radio-frequency (RF) or infrared fields, as well as their preparation. DNA dendrimers bound with particles containing elements that heat in the presence of an electromagnetic field are suitable for use as devices capable of thermally ablating targets in in-vitro, ex-vivo and in-vivo applications. Significant increases of thermal ablation efficiency of target cells and tissues may be achieved via the use of DNA dendrimers containing multiple metallic nanoparticles per dendrimer molecule, particularly when the DNA dendrimer also contains a targeting device capable of directing the particle laden dendrimer to the surface of the desired target cell and tissues.

Applications of dendrimer directed thermal ablation according to the invention include 1) thermal ablation of diseased cells and tissues, e.g. cancerous cells either concentrated in tumors, metastasized cells spread throughout the body, or circulating cancerous cells as found in leukemias and other leukoproliferative disorders; 2) ablation of cells and tissues that would otherwise be surgically removed; 3) ablation of microorganisms in-vivo that are resistant to other therapeutic treatments (e.g. antibiotic resistant bacteria and other organisms); 4) ex vivo treatment of cells, tissues and organs prior to transplant, including transplant organs, blood products and bone marrow; and 5) other applications where proximity of a thermally responsive nano-device would be of benefit, including a wide range of in-vivo, ex-vivo and in-vitro processes.

The stability of the DNA dendrimer in the presence of living cells in-vitro, ex-vivo and in-vivo, has also been a serious concern given the potential for degradation of the DNA dendrimer by endogenous or exogenous protein nucleases. For example, prior data had indicated that DNA dendrimers did not survive intact for more than a few minutes in the presence of fresh human or animal serum. Unexpectedly, we found that DNA dendrimers that contained the intercalation cross-linking agent psoralen and that also contained attached label (and other) moieties (e.g. FITC) and proteins (e.g. targeting antibodies) were extraordinarily resistant to nuclease degradation in the presence of human or animal serum samples. See WO 2010/017544, incorporated by reference in its entirety. This was a surprising result as non-dendritic ssDNA or dsDNA molecules are typically degraded rather quickly in the presence of nucleases.

While targets for thermal ablation primarily include animate and biological objects, there are possible benefits for using dendrimer for thermal ablation of inanimate objects where exposure to high temperatures contained within the very small volume of a particle laden DNA dendrimer would have added value to a particular process. A wide range of nanomaterials may benefit from the use of thermal ablation via targeted DNA dendrimers containing nanoparticles, including applications in electronics and the manufacture of various nano-particle containing materials.

In one aspect, the invention relates to DNA dendrimers linked to one or more targeting moieties and to one or more radiation absorbing nanoparticles which can be heated in situ by electromagnetic energy. In a particular embodiment, the radiation absorbing nanoparticle associated with the DNA dendrimer can be heated to at least 40° C., 40-50° C., 50-60° C., 60-70° C. or even 70-80° C., by externally applied electromagnetic radiation. In a further particular embodiment, the targeting moiety is an antibody which recognizes and binds to a tumor-specific or tumor-associated antigen on a cell surface, such as a receptor. In yet a further particular embodiment, the radiation absorbing nanoparticle is a gold nanoparticle. In yet a further embodiment, the electromagnetic energy is RF radiation.

In another aspect, the invention relates to methods for making thermal ablation DNA dendrimers wherein the methods comprise covalently binding one more radiation absorbing nanoparticles and one or more targeting moieties to a DNA dendrimer. In a specific aspect, capture oligonucleotides may be appended to the DNA dendrimer arms and complementary oligonucleotides conjugated to the nanoparticles may be hybridized to the capture oligonucleotides. The targeting moieties may also be covalently bound to oligonucleotides which are complementary to a capture sequence on the DNA dendrimer arms and hybridized to the capture oligonucleotides. Following hybridization to the capture oligonucleotides either or both of the complementary oligonucleotides may optionally be cross-linked to the capture oligonucleotides of the DNA dendrimer.

In a further aspect, the invention provides methods for thermal ablation of cells or tissues using the thermal ablation DNA dendrimers and pharmaceutical compositions. For example, cells or tissues may be contacted with a pharmaceutical composition comprising thermal ablation DNA dendrimers which target a feature on the cell surface under conditions which allow the targeting moiety of the DNA dendrimer to bind to a complementary target on the cell or tissue. The cells or tissues with the bound thermal ablation DNA dendrimers are then exposed to externally applied electromagnetic radiation, such as RF radiation, for a time and at a power sufficient to cause the attached nanoparticles to emit heat. Preferably, the nanoparticles are exposed to electromagnetic radiation, such as RF radiation, such that the nanoparticles generate heat of at least 40° C., 40-50° C., 50-60° C., 60-70° C., or 70-80° C., thereby resulting in thermal ablation of cells or tissues bound to the thermal ablation DNA dendrimers. In a specific embodiment, cells in in vitro cell culture are contacted with the thermal ablation DNA dendrimer and exposed to electromagnetic radiation, such as RF radiation, from an external source directed at the cell culture to achieve thermal ablation of the targeted cells. In an alternative specific embodiment, cells or tissues are contacted in vivo or ex vivo with the thermal ablation DNA dendrimers and exposed to electromagnetic radiation, such as RF radiation, from an external source directed at the cells or tissues bound to the thermal ablation DNA dendrimers to achieve thermal ablation of the cells or tissues. Examples of cells and tissues for in vivo or ex vivo thermal ablation include tumors and biological materials for transplantation.

### BRIEF DESCRIPTION OF THE DRAWINGS

FIG. 1 illustrates various methods for hybridizing oligonucleotides labeled with radiation absorbing nanoparticles to the extension oligonucleotides and arms of DNA dendrimers.

FIG. 2 illustrates attachment of targeting moieties to the DNA dendrimers with attached radiation absorbing nanoparticles shown in FIG. 1.

FIG. 3 illustrates hybridization of oligonucleotides labeled with radiation absorbing nanoparticles to the extension oligonucleotides and arms of a non-spherical DNA dendrimer.

FIG. 4 illustrates hybridization of oligonucleotides labeled with radiation absorbing nanoparticles to the extension oligonucleotides of a DNA dendrimer monomer.

FIG. 5 illustrates hybridization of oligonucleotides labeled with radiation absorbing nanoparticles to a linear DNA dendrimer.

The following examples are not intended to be limiting, and modifications and variations thereto are well within the scope of those skilled in the art.

### DETAILED DESCRIPTION

As used herein, the term “thermal ablation DNA dendrimer” refers to a DNA dendrimer linked covalently or noncovalently to a) one or more targeting moieties and b) to one or more radiation absorbing nanoparticles.

As used herein, the term “targeting moiety” or “targeting device” refers to a molecule which recognizes and binds to a complementary molecule on the surface of a cell or tissue. Non-limiting examples of targeting moieties include antibodies, antibody fragments, binding proteins and peptides, receptors and ligands for receptors.

As used herein, the term “radiation absorbing nanoparticles” refers to nanoparticles which absorb electromagnetic radiation (including as examples infrared, near-infrared (NIR) and radio-frequency (RF) radiation) and convert the absorbed energy to released heat which can be used to create localized hyperthermia.

As used herein, the term “external source” or “externally applied” with respect to exposure to electromagnetic radiation refers to directing the electromagnetic radiation toward the cell or tissue target from outside the body of a patient or from outside of a cell or tissue culture. This method of delivering electromagnetic radiation to the desired site for biomedical purposes is to be distinguished from conventional methods in which such radiation is delivered to a target site via a needle or probe implanted at the site.

As used herein, the term “arms” with respect to DNA dendrimers refers to the single-stranded ends of the monomers which form the DNA dendrimer and are available for hybridization or attachment of functional molecules such as detection, delivery and capture agents.

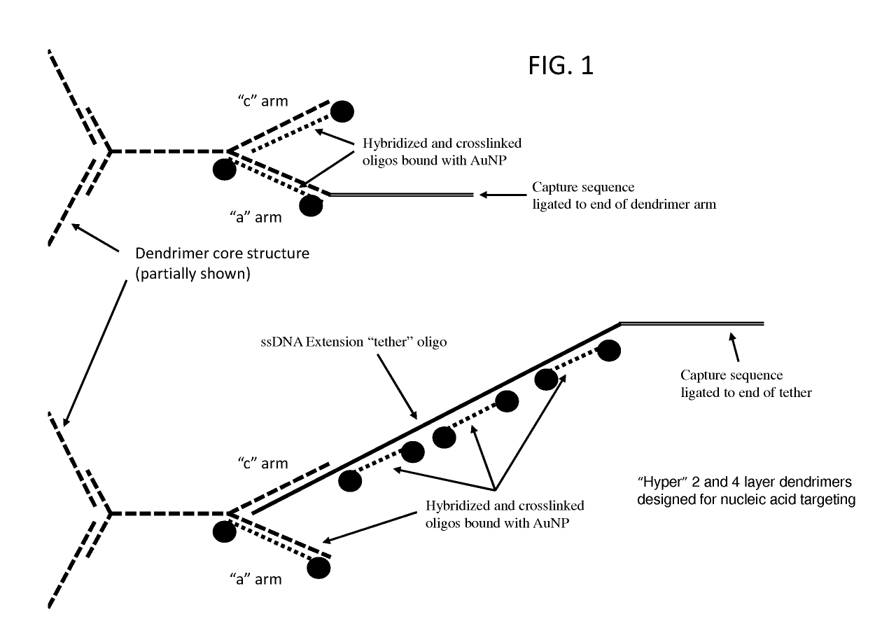

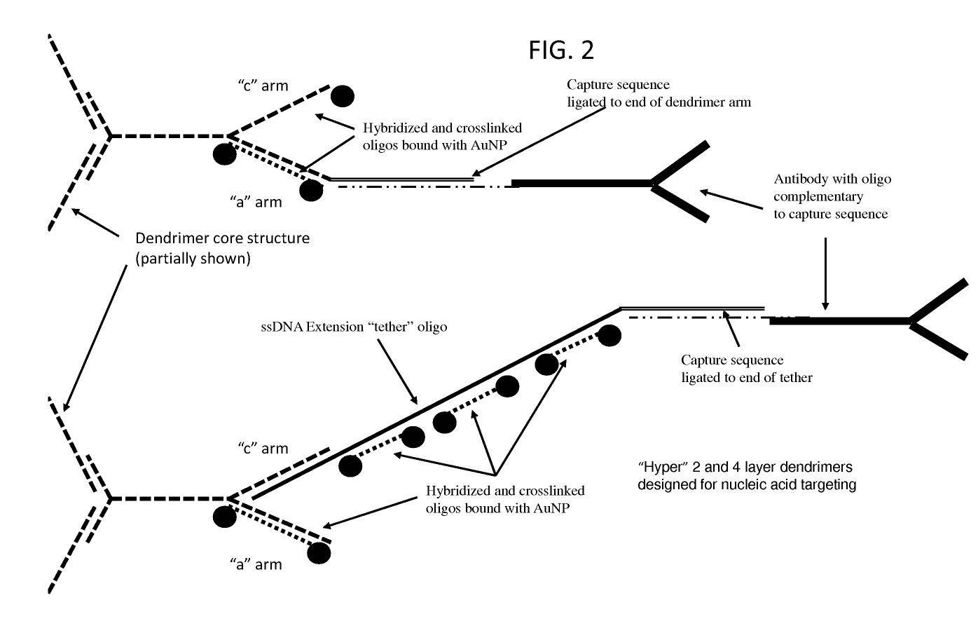

In a first embodiment, the invention provides thermal ablation DNA dendrimers. The thermal ablation dendrimers comprise one or more targeting moieties linked to one or more dendrimer arms and one or more radiation absorbing nanoparticles also linked to one or more dendrimer arms. Either or both of the targeting moieties and the nanoparticles may be covalently linked directly to the DNA dendrimer. Alternatively, either or both of the targeting moieties and the nanoparticles may be linked to the DNA dendrimer by hybridization of an oligonucleotide carrying the targeting moiety or the nanoparticle to the DNA dendrimer, as shown in FIG. 1 and FIG. 2. In a further alternative aspect, either or both of the targeting moieties and the nanoparticles may be linked to the DNA dendrimer by hybridization to the DNA dendrimer of a carrier oligonucleotide conjugated to the targeting moieties or the nanoparticles. The carrier oligonucleotide may optionally be crosslinked to the DNA dendrimer.

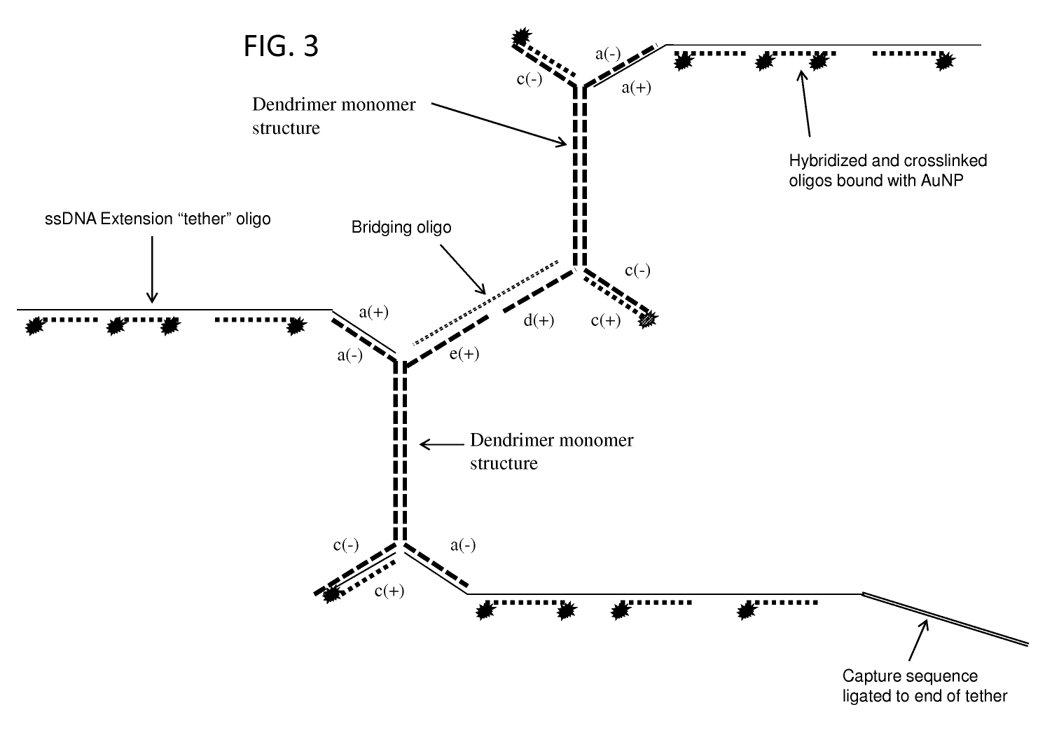

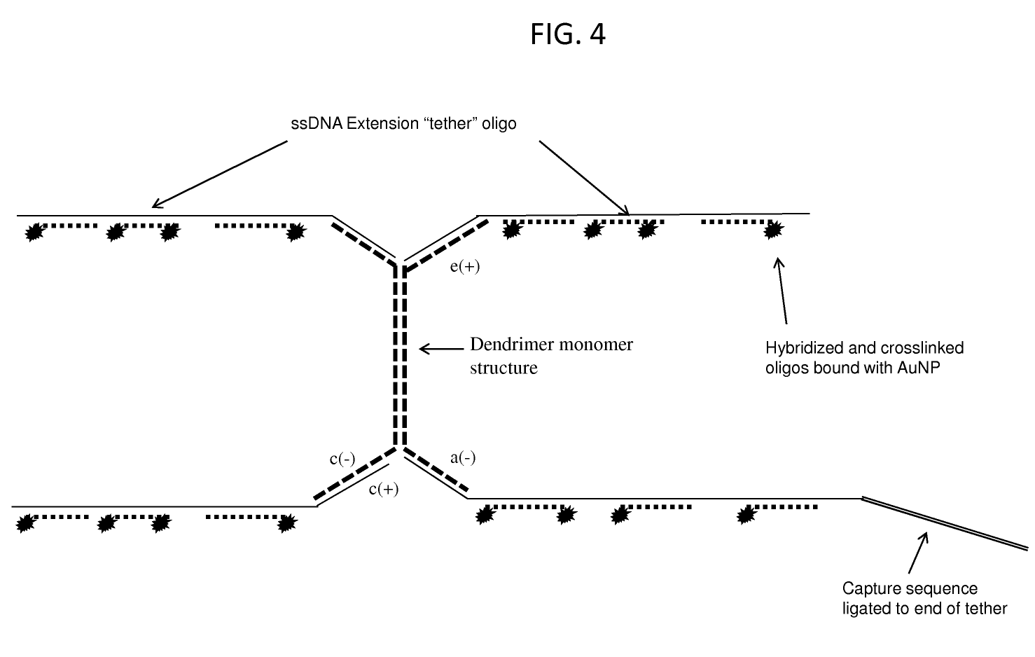

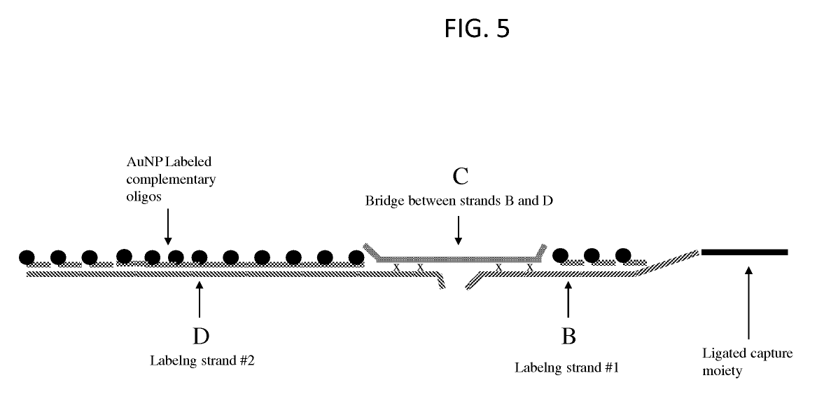

The DNA dendrimer component of the thermal ablation DNA dendrimers may be any DNA dendrimer known in the art, for example as described in U.S. Pat. Nos. 5,175,270, 5,484,904, 5,487,973, 6,110,687 and 6,274,723, and include nonspherical, and three-dimensional or spherical DNA dendrimers. The three-dimensional or spherical DNA dendrimer may be a one-layer, two-layer, three-layer or four-layer DNA dendrimer but may also comprise more than four layers. In a first specific example, the DNA dendrimer comprises at least four-layers. Nonspherical and linear DNA dendrimers provide a more compact structure and a higher ratio of nanoparticles to dendrimer mass than three-dimensional DNA dendrimers, which may improve uptake in tissues and enhance efficiency of thermal ablation. For example, FIG. 3, FIG. 4 and FIG. 5 show various types of nonspherical and linear DNA dendrimers hybridized to oligonucleotides labeled with radiation absorbing nanoparticles. In some cases approximately 30-35 nanoparticles can be linked to a linear dimer DNA dendrimer which consists of only four strands.

The number of layers in a three-dimensional DNA dendrimer or the number of strands in a linear DNA dendrimer determines the size of the DNA dendrimer. This can be used to select and optimize the thermal ablation DNA dendrimers of the invention, as the size impacts the ability of the thermal ablation DNA dendrimer to access the thermal ablation target site and to avoid phagocytic clearance when administered parenterally for in vivo applications. The practitioner may therefore construct a DNA dendrimer of a selected size, based on the number of layers or strands, to obtain a desired half-life on parenteral administration and delivery of the desired amount of thermal ablation capability. For example, a four-layer DNA dendrimer is typically approximately 170 nm in diameter, and would not be expected to be particularly susceptible to phagocytic clearance. It is also large enough to carry a substantial number of targeting moieties and a substantial number of radiation absorbing nanoparticles to provide thermal ablation efficacy and efficient targeting of the cell or tissue of interest.

The monomers of the DNA dendrimer core may be crosslinked, for example with psoralen, to ensure stability during in vivo use. However, DNA dendrimers constructed without crosslinking (i.e., based only on hybridization of the arms of the monomers) are stable at 37° C. and are therefore expected to maintain hybridization in vivo. In addition, the binding sites of the arms are typically 31 nucleotides or more in length with an estimated Tₘ of 65° C. and the “waist” of the monomers is at least 50 nucleotides in length with an estimated Tₘ of greater than 80-90° C. For these reasons, if crosslinking reagents are considered to be undesirable for in vivo use, the thermal ablation DNA dendrimers of the invention constructed by hybridization alone are expected to be stable at in vivo temperatures.

The radiation absorbing nanoparticles linked to the arms of the thermal ablation DNA dendrimers may be present in any number suitable to produce the desired efficacy of thermal ablation in a selected application. It is understood that the number of nanoparticles per dendrimer will be limited by the size of the DNA dendrimer to which they are linked, but the size of the DNA dendrimer can be modified appropriately as discussed above. It is also understood that at least some of the available dendrimer arms may remain free for linkage of the targeting moiety. As an example, a thermal ablation DNA dendrimer may comprise 15-1200 radiation absorbing nanoparticles, 25-500 radiation absorbing nanoparticles, 50-350 radiation absorbing nanoparticles, 100-500 radiation absorbing nanoparticles, 200-400 radiation absorbing nanoparticles, or about 300 radiation absorbing nanoparticles. The radiation absorbing nanoparticles are typically about 5-20 nm, 5 nm, 10 nm, 15 nm, or 20 nm in size.

The targeting moieties linked to the arms of the thermal ablation DNA dendrimers may be present in any number suitable to obtain the desired degree of binding to the targeted tissue or cell in a selected application. It is understood that the number of targeting moieties per dendrimer will be limited by the size of the DNA dendrimer to which they are linked, but the size of the DNA dendrimer can be modified appropriately as discussed above. It is similarly understood that at least some of the available dendrimer arms may remain free for linkage of the radiation absorbing nanoparticles. As an example, a thermal ablation DNA dendrimer may comprise a number of radiation absorbing nanoparticle sufficient to provide in situ heating to at least 40° C., 40-50° C., 50-60° C., 60-70° C. or 70-80° C., using externally applied electromagnetic radiation, such as RF radiation. Thermal ablation DNA dendrimers according to the invention may have, on average, from less than one to greater than 100, from 2 to 120, from 15 to 50 or from 30 to 35 targeting moieties linked to the arms, depending on the size and structure (linear or three-dimensional) of the dendrimer. In a specific example, about 25 targeting moieties can be linked to a four-layer DNA dendrimer.

In a second embodiment, the invention provides methods of making thermal ablation DNA dendrimers. Methods for construction of the DNA dendrimers are referenced above. Targeting moieties and radiation absorbing nanoparticles can then be linked to the arms of the DNA dendrimer. In a first example, the targeting moieties and/or the radiation absorbing nanoparticles are linked directly to the arms of the DNA dendrimer via chemical conjugation as is known in the art. However, in a second example the targeting moieties and/or the radiation absorbing nanoparticles are linked to the DNA dendrimer via a capture oligonucleotide associated with the arm of the DNA dendrimer. The capture oligonucleotide is generally associated with the terminus of the DNA dendrimer arm. Typically it is ligated to the terminus of the arm of the DNA dendrimer, but it may also be hybridized to the terminus and optionally crosslinked thereto or associated with the arm by use of an extension oligonucleotide as described below. The capture oligonucleotide provides a specific, defined sequence present in a defined quantity for hybridization to a complementary carrier oligonucleotide linked to the targeting moiety or the nanoparticle. The capture oligonucleotide also provides a means for controlling the number of targeting moieties and nanoparticles linked to the DNA dendrimer, as the carrier oligonucleotides can be hybridized to the capture oligonucleotide at a defined concentration which results in the desired number of DNA dendrimer arms being occupied by each component. The hybridization concentration and volume of the carrier oligonucleotides can thus be varied to adjust the number of nanoparticles and targeting moieties per dendrimer.

Upon hybridization of the complementary carrier oligonucleotide, the targeting moiety or nanoparticle becomes linked to the arm of the DNA dendrimer through Watson-Crick base pairing. FIG. 2 shows linking of a targeting antibody to the DNA dendrimer via a carrier oligonucleotide hybridized to the capture sequence linked to the arm of the dendrimer. Optionally, the hybridized carrier oligonucleotide may then be covalently bound to the arm of the DNA dendrimer, for example by crosslinking the hybridized oligonucleotides. One such method involves incorporating a DNA-DNA crosslinking agent such as psoralen (e.g., 2,4,8-trimethyl psoralen) into the oligonucleotide and exposing the hybridized oligonucleotides to UV light. Targeting moieties may be conjugated to carrier oligonucleotides using condensation chemistry for linking proteins or peptides to oligonucleotides as is known in the art, for example using chemistries available from Solulink, Inc. (San Diego, Calif.). Radiation absorbing nanoparticles may be conjugated to carrier oligonucleotides, for example using the methods described by David J. Javier, et al. 2008 Bioconjugate Chem., 19(6):1309-1312. Briefly, an HPLC purified oligonucleotide is reduced with TCEP (tris(2-carboxyethyl)phosphine hydrochloride) and added to a solution of colloidal gold nanoparticles. The conjugate is aged with increasing concentrations of PBS until reaching a 1× concentration of PBS. Unreacted capture oligonucleotide is removed by centrifugation. When the targeting moieties and/or radiation absorbing nanoparticles are conjugated to carrier oligonucleotide it is beneficial for in vivo use to select the carrier and capture oligonucleotide sequences and length such that the duplex has a Tₘ of at least 40° C., 40-70° C., 50-70° C. or 60-70° C. to prevent disassociation in vivo.

In a specific embodiment, the targeting moiety may be linked to the DNA dendrimer via a carrier oligonucleotide which is complementary to the capture oligonucleotide, as described above, and the nanoparticles may be directly conjugated to the DNA of the dendrimer arms or hybridized to the arms via hybridization of complementary oligonucleotides linked to the nanoparticles (see FIG. 1, top). In this embodiment, the hybridization of the carrier oligonucleotide with the targeting moiety to the terminal sequences of the dendrimer arms (extended by addition of the capture oligonucleotide) leaves sufficient space on the interior segment of the same arm to link radiation absorbing nanoparticles. The nanoparticles may be linked, for example, by biotinylating the DNA of the interior segment and binding streptavidin-coated nanoparticles to the biotin.

In a further specific embodiment, the free arms of the DNA dendrimer may be extended by hybridization to extension oligonucleotides (see FIG. 1 and FIG. 2, bottom). The capture oligonucleotide may be ligated or otherwise linked to the termini of the extension oligonucleotides for hybridization to the carrier oligonucleotide/targeting moiety, or the targeting moiety may be linked directly to the extension oligonucleotide. The extension oligonucleotide can be used to place the targeting moiety at a distance further from the core of the DNA dendrimer than the capture oligonucleotide alone, thus reducing steric hindrance when multiple thermal ablation DNA dendrimesr bind to a cell or tissue. That is, while the capture oligonucleotide is relatively short (on the order of 25-40 nucleotides long), the extension oligonucleotide can be of any length necessary to reduce or overcome steric hindrance in a particular application. For example, extension oligonucleotides may be 60-140 nucleotides long, 80-130 nucleotides long, 100-125 nucleotides long or 124 nucleotides long. As an example, a 124 nucleotide extension oligonucleotide may provide 85-90 nucleotides of extension after hybridization to the dendrimer arm. Also, by using extension oligonucleotides the unhybridized segment of the extension oligonucleotide between the arm of the dendrimer and the capture oligonucleotide is available for hybridization to additional labeled or nanoparticle-linked oligonucleotides (see FIG. 1 and FIG. 2, bottom).

In a first aspect, the extension oligonucleotides may have a defined nucleotide sequence. The defined nucleotide sequence may be any selected sequence but is preferably an abiotic sequence. In alternative aspects, the extension oligonucleotides may have a homopolymeric sequence (for example poly(dT), poly(dA), poly(dG) or poly(dC)) or they may comprise a repeat sequence. If the extension oligonucleotides comprise a repeat sequence, the repeat will generally be 2-15 nucleotides, 2-12 nucleotides, 2-10 nucleotides or 2-8 nucleotides in length. However, it is to be understood that the repeat sequence may be of any length provided that it appears at least twice in the extension oligonucleotide. Useful methods for preparing optimally labeled oligonucleotides using repeat sequences can be found in U.S. Pat. Nos. 6,072,043 and 6,046,038.

In a third embodiment, the invention provides methods of using thermal ablation DNA dendrimers for targeted thermal ablation of selected cells or tissues. In general, the time of exposure and power of electromagnetic radiation, such as RF radiation, will be selected based on the desired outcome, the particular properties of the cells or tissues being targeted for thermal ablation, and the heat-generating and cell targeting capabilities of the selected thermal ablation DNA dendrimer. Cells or tissues bound to the thermal ablation DNA dendrimers may be exposed to the electromagnetic field from an external source at a power of from 1 W to 2000 W, 10 W to 1500 W, 10 W to 200 W, or about 50 W for 1 min. to 2 hrs. or until the desired degree of thermal ablation is achieved. In a specific embodiment of these methods, cells or tissues are contacted with the thermal ablation DNA dendrimers in vitro, for example in cell or tissue cultures. In a further specific embodiment of these methods, cells or tissues are contacted with the thermal ablation DNA dendrimers in vivo, for example by parenteral administration to a human. In one example of in vivo use, the thermal ablation DNA dendrimers may be administered intravenously or directly into a group of cells or a tissue through a needle or catheter. Such administration may be as a bolus injection or continuous infusion prior to exposure to the external electromagnetic field. Following administration it will generally be desirable to allow sufficient time for the thermal ablation DNA dendrimers to bind to the targeted cells or tissues before exposure to the electromagnetic field. If the thermal ablation DNA dendrimers are administered intravenously, the time required for binding to the targeted cells or tissues will be longer than for direct injection due to the time required for circulation of the dendrimers and accumulation at the target site.

When used for targeted thermal ablation of selected cells or tissues, the DNA dendrimers may be constructed prior to contacting the cells or tissues selected for thermal ablation. That is, if the thermal ablation is to be conducted in vivo the thermal ablation DNA dendrimers may be fully assembled (dendrimer linked to radiation absorbing nanoparticle and targeting moiety) prior to administration to the patient. If thermal ablation is to be conducted in vitro or ex vivo the thermal ablation DNA dendrimers may be fully assembled prior to contacting the cells or tissues for thermal ablation. Alternatively, the thermal ablation DNA dendrimers may constructed in vivo by separately administering the components of the thermal ablation DNA dendrimers and allowing post-administration assembly on the targeted cells or tissues. For example, the DNA dendrimers (without linked radiation absorbing nanoparticles or targeting moieties) may be administered, followed by the targeting moiety and the radiation absorbing nanoparticles in either order or simultaneously. In another example, the targeting moiety may be administered, followed by the DNA dendrimers and the radiation absorbing nanoparticles in either order or simultaneously. This sequential assembly approach may also be applied to in vitro and ex vivo uses.

In specific methods of use, the thermal ablation DNA dendrimers of the invention may be used as described for ablation of tumors such as hepatic cancers, gastrointestinal cancers, breast cancers, pancreatic cancers, lung cancers, prostate cancers, and any other localized solid tumor which is targetable by DNA dendrimers and amenable to external electromagnetic field exposure. In further specific methods of use, the thermal ablation DNA dendrimers of the invention may be used as described for ablation of circulating tumor cells, such as leukemia or lymphoma cells, or for thermal ablation of foci of cancer metastases. In addition, the thermal ablation DNA dendrimers of the invention may be used for ablation of microorganisms such as Borrelia, Staphylococcus aureus (including methicillin-resistant S. aureus, MRSA), and vancomycin resistant bacteria. In ex vivo applications, biological materials such as organs, cells and tissues for transplantation may be treated using the thermal ablation DNA dendrimers to ablate undesirable cells such as cancer cells prior to transplant. In a specific example, the thermal ablation DNA dendrimers of the invention may be used in autologous bone marrow transplantation to ablate cancer cells from the aspirated bone marrow of a cancer patient prior to reintroducing the bone marrow to the patient.

In a fourth embodiment the invention provides pharmaceutical compositions comprising the thermal ablation DNA dendrimers for use in the described methods of treatment of cancers and tumors. Such pharmaceutical compositions will generally be formulated for either systemic or local parenteral administration, for example for intravenous administration or for injection directly into the site to be treated using a syringe or catheter. The pharmaceutical compositions will generally further include at least one pharmaceutically acceptable carrier or excipient as is known in the art. See, e.g., “Handbook of Pharmaceutical Excipients, 4^{th} ed. (2003) Raymond C. Crowe, et al. eds. Pharmaceutical Press, Chicago. Pharmaceutically acceptable carriers and excipients include stabilizing agents, buffering agents, solubilizing agents, etc. such as starches, cellulose derivatives, polyethylene glycols, calcium carbonate, calcium phosphate, sodium phosphate, sugars and the like. Formulations of appropriate pharmaceutical compositions may be found in “Remington's Pharmaceutical Sciences,” Mack Publishing Co., Easton, Pa. The thermal ablation DNA dendrimers of the invention are soluble in aqueous solution, which allows preparation of the pharmaceutical compositions in physiologically compatible aqueous buffers such as Hank's solution, Ringer's solution, normal saline or physiological salt buffers.

In any of the foregoing embodiments, the targeting moieties linked to the thermal ablation DNA dendrimers may be any moiety which specifically binds to a selected target on the cell or tissue of interest for thermal ablation. Specific binding to a selected target includes not only exclusive binding to a cell or tissue of interest for thermal ablation, but also differential binding between a cell or tissue of interest for thermal ablation and a cell or tissue which is not targeted for thermal ablation. For example, cells or tissues exhibiting a higher density of the target as compared to other cells or tissues exhibiting the same target may be selectively ablated based on the greater amount of binding of the DNA dendrimers and therefore the greater exposure to radiation absorbing nanoparticles. Targeting moieties include proteins, peptides and aptamers. In a specific embodiment such targeting moieties may be antibodies or antibody fragments (including Fab, F(ab)₂, scFv, diabodies, and minibodies). The antibodies or antibody fragments are directed to a binding partner on the surface of the cell or tissue of interest for thermal ablation, preferably a specific binding partner that distinguishes the target cell or tissue from other cells or tissues not targeted for thermal ablation. If the cell or tissue targeted for thermal ablation is a malignant cell or tumor the antibody or antibody fragment may bind a tumor-specific or tumor-associated antigen, for example alphafetoprotein, carcinoembryonic antigen, CA-125, MUC-1, epithelial tumor antigen, tyrosinase, melanoma-associated antigen, or ras or p53 gene products. The targeting moiety antibody or antibody fragment may alternatively bind to a receptor on the surface of the cell or tissue targeted for thermal ablation, for example EGFR or HER2. Peptides or proteins on the surface of the cell or tissue may also be targeted by antibodies or antibody fragments, for example LHRH peptides or integrins. Alternatively, in a further specific embodiment, the targeting moiety linked to the thermal ablation DNA dendrimer may be a ligand for a receptor on the cell or tissue surface. Such ligands are generally peptides or small proteins, for example, TNF-α, lymphotoxin, transforming growth factor-β, insulin, insulin-like growth factor-1, VEGF, PDGF, EGF, FGF, TSH, and ACTH.

In any of the foregoing embodiments, the radiation absorbing nanoparticles linked to the thermal ablation DNA dendrimers may be of any composition which absorbs electromagnetic energy, such as RF energy, and releases it as heat, including metallic nanoparticles and carbon-based nanoparticles. Such radiation absorbing nanoparticles may be in the form of nanospheres, nanorods, nanoshells, nanocages, nanotubes, or surface-enhanced Raman scattering (SERS) nanoparticles as is known in the art. In specific examples, the nanoparticles comprise carbon, silver or gold. In a further specific example, the nanoparticles comprise gold, which has the advantage of prior use in medical applications and therefore demonstrated medical acceptability.

In any of the foregoing embodiments, the thermal ablation DNA dendrimers of the invention may further include a tracking label linked to the dendrimer arms via any of the methods and structures herein described. Tracking labels allow the location of the thermal ablation DNA dendrimer to be detected and monitored, which is particularly useful for in vivo applications where time is required after administration to allow the dendrimers to accumulate at the desired targeted site. By including a tracking label, the user can monitor accumulation of the dendrimer over time and determine the appropriate time to apply the external electromagnetic field to the target site. Useful tracking labels include fluorescent labels such as near-infrared fluorescent dyes (for optical imaging), radioactive labels such as ¹⁸F (a radiotracer used in PET scanning), and contrast agents such as gandolinium (a paramagnetic material used in MRI).

In certain embodiments the DNA dendrimers of the invention, comprising at least one targeting moiety and at least one metallic radiation absorbing nanoparticle, may also be used as imaging agents either in vivo, ex vivo or in vitro. In this embodiment the DNA dendrimers are administered to a patient, cell culture or tissue and allowed to bind their target on the cell or tissue of interest for imaging. Instead of exposing the bound DNA dendrimers to an external electromagnetic field to produce heat, the location of the bound DNA dendrimers is detected by imaging technologies which detect the bound radiation absorbing nanoparticles associated with the DNA dendrimer. For example, the imaging DNA dendrimers may be used as molecular-specific contrast agents for reflective imaging (Javier, et al., supra), photothermal interference contrast, dark-field imaging, scanning electron microscopy, fluorescence microscopy, photoacoustic tomography, optical coherence tomography, magnetic resonance imaging, and Raman spectroscopy (reviewed in Cai, et al., Nanotechnology, Science and Applications 2008:I 17-32).

When used for imaging of cells or tissues, the DNA dendrimers may be constructed prior to contacting the cells or tissues. That is, if imaging is to be conducted in vivo the DNA dendrimers may be fully assembled (dendrimer linked to radiation absorbing nanoparticle and targeting moiety) prior to administration to the patient. If imaging is to be conducted in vitro or ex vivo the DNA dendrimers may be fully assembled prior to contacting the cells or tissues. Alternatively, the DNA dendrimers may constructed in vivo by separately administering the components of the DNA dendrimers and allowing post-administration assembly on the targeted cells or tissues. For example, the DNA dendrimers (without linked radiation absorbing nanoparticles or targeting moieties) may be administered, followed by the targeting moiety and the radiation absorbing nanoparticles in either order or simultaneously. In another example, the targeting moiety may be administered, followed by the DNA dendrimers and the radiation absorbing nanoparticles in either order or simultaneously. This sequential assembly approach may also be applied to in vitro and ex vivo uses.

### EXAMPLES

### Example 1

### Manufacture of a DNA Dendrimer Containing a Capture Oligonucleotide

DNA dendrimers were manufactured as previously disclosed (see, e.g., U.S. Pat. Nos. 5,175,270, 5,484,904, 5,487,973, 6,110,687 and 6,274,723, each of which is incorporated by reference in its entirety). Briefly, a DNA dendrimer was constructed from DNA monomers, each of which is made from two DNA strands that share a region of sequence complementarity located in the central portion of each strand. When the two strands anneal to form the monomer the resulting structure can be described as having a central double-stranded “waist” bordered by four single-stranded “arms”. This waist-plus-arms structure comprises the basic 3DNA® monomer. The single-stranded arms at the ends of each of the five monomer types were designed to interact with one another in precise and specific ways. Base-pairing between the arms of complementary monomers allows directed assembly of the dendrimer through sequential addition of monomer layers. Assembly of each layer of the dendrimer included a cross-linking process where the strands of DNA were covalently bonded to each other, thereby forming a completely covalent molecule impervious to denaturing conditions that otherwise would cause deformation of the dendrimer structure. In addition, 38 base oligonucleotides that serve as complementary capture oligos were ligated to the 5′ ends of available dendrimer arms via a simple T4 DNA ligase dependent ligation reaction, as follows:

The 38 base DNA capture oligonucleotides were covalently attached to the ends of the dendrimer arms via a simple nucleic acid ligation reaction utilizing a “bridging oligonucleotide” that overlaps adjacent portions of the dendrimer arm and the capture oligonucleotide, thereby bridging the capture oligonucleotide to the end of the dendrimer arm. The bridging oligonucleotide overlapped at least 5 bases of each of the adjacent dendrimer arm and capture oligonucleotide sequences to facilitate the ligation activity of a nucleic acid ligase enzyme (preferably T4 DNA ligase enzyme), with at least 7 bases of overlap of each sequence preferred. The bridging oligo may also serve as a nucleic acid blocker for its complementary sequences when the dendrimer is used for specific targeting of non-dendrimer nucleic acids or other molecules.

The following components were added to a microfuge tube:

| | | |
|---|---|---|
| 4 layer DNA dendrimer (500 ng/μL) in 1X TE | 5.4 | μL (2680 ng) |
| buffer | | |
| a(−)LIG-BR7 Bridging oligo (14mer) (50 ng/μL) | 2.7 | μL (134 ng) |
| 10X Ligase buffer | 10.2 | μL |
| Nuclease free water | 81.7 | μL |
| Cap03 capture oligo (38mer) (50 ng/μL) | 4.0 | μL (200 ng) |
| T4 DNA Ligase (1 U/μL) | 10.0 | μL (10 units) |

The first four reactants were added together, heated to 65° C. and cooled to room temperature. The 5^{th} and 6^{th} reactants were then added and incubated for 45 minutes. The ligation reaction was stopped by adding 2.8 μL of 0.5M EDTA solution. Non-ligated oligonucleotide were removed via the use of a size exclusion spin column. The dendrimer ligated with the Cap03 sequence was adjusted to 50 ng/μL in 1×TE buffer for use in subsequent steps to attach gold nanoparticles and antibody to the DNA dendrimer.

### Example 2

### Attachment of Gold Nanoparticles (AuNP) Via Biotin Labeled Oligonucleotides and Targeting Antibodies Via Carrier Oligonucleotides to the DNA Dendrimer

The following components were added to a microfuge tube:

| | |
|---|---|
| 4 layer DNA dendrimer with ligated Cap03 sequence | 50.0 μL |
| (50 ng/μL) | |
| c(+) oligo 3′ end labeled with biotin (500 ng/μL) | 2.6 μL |
| a(+) oligo 5′ end labeled with biotin (500 ng/μL) | 2.6 μL |
| 5M NaCl | 4.0 μL |
| 2,4,8 trimethyl psoralen saturated in ethanol | 7.0 μL |

The above reactants are added together, mixed well, placed into a container of water at 65° C. and slow cooled to 42° C. Exposure to UV light (320-400 nm) for 10 minutes (×2) initiates a cross-linking event covalently binding the biotinylated oligos to the arms of the DNA dendrimer. Non-cross-linked oligonucleotides are removed via the use of a size exclusion spin column. Small quantities of fluorescent c(+) and/or a(+) oligos are added to some preparations to provide fluorescent labels to assist in tracking dendrimers binding to cellular surfaces.

Targeting antibodies were bound to DNA dendrimers by first covalently conjugating a DNA oligonucleotide to either a complete antibody or an antibody fragment (Fab or Fab′(2)) using standard cross-linking condensation conjugation chemistry, followed by hybridizing the antibody-bound oligonucleotide to a complementary sequence on the arms of the dendrimer. This hybridization comprised 31 base pairs with a melting temperature of greater than 65° C., thereby providing a stable complex of dendrimer bound with antibody at physiological temperatures and conditions. Simultaneous with the binding of the targeting antibody to the dendrimer, streptavidin-AuNP was added at appropriate stoicheometry and allowed to bind to the biotin moieties previously attached to the dendrimer structure.

The following components were added to a microfuge tube:

| | |
|---|---|
| 4 layer biotinylated DNA dendrimer with ligated Cap03 | 50.0 μL |
| sequence | |
| 50% ethelyene glycol in PBS or equivalent | 125.0 μL |
| (e.g. Superfreeze, Pierce Fine Chemicals) | |
| 1X Phosphate Buffered Saline (PBS) | 57.0 μL |
| 5M NaCl | 4.3 μL |
| Antibody (anti-human HLA Class I Mab) with anti-Cap03 | 13.7 μL |
| oligo previously covalently bound (10 ng/μL as oligo) | |
| Streptavidin-AuNP (20 nm) (BBI Ltd.) (1.7 × 10¹² | 19.5 μL |
| AuNP per mL) | |

The above reactants are combined, gently mixed and incubated at 37° C. for 30 minutes. This formulation is stable at 4° C. for at least six months.

Using the above biotin-streptavidin linking methods, the following gold-nanoparticle conjugated DNA dendrimers have been produced with 5 nm, 10 nm, 15 nm, and 20 nm gold-nanoparticles:

| | # Nanogold Labels Per |
|---|---|
| Dendrimer Type | Dendrimer |
| 2-layer | 60 |
| 2-layer | 30 |
| 2-layer | 15 |
| 2-layer (extended arms) | 150 |
| 2-layer (extended arms) | 120 |
| 2-layer (extended arms) | 60 |
| 2-layer (extended arms) | 30 |
| 4-layer | 240 |
| 4-layer | 480 |
| 4-layer | 240 |
| 4-layer | 120 |
| 4-layer | 60 |
| 4-layer (extended arms) | 1200 |
| 4-layer (extended arms) | 720 |
| 4-layer (extended arms) | 480 |
| 4-layer (extended arms) | 120 |

### Example 3

### Attachment of AuNP Via Oligonucleotide Hybridization and Targeting Antibodies Via Carrier Oligonucleotide to the DNA Dendrimer

Small DNA or RNA oligonucleotides (and other biochemical analogs) conjugated with gold nanoparticles (or other label moieties) are hybridized to the dendrimer “arm” single stranded nucleic acid sequences on the periphery of the dendrimer matrix structure. These labeled oligonucleotides may be bound via typical Watson-Crick base pairing only, or may be further covalently crosslinked to the dendrimer structure via the use of UV activated psoralen intercalators which form covalent bonds between thymines on adjacent hybridized DNA strands.

The following components were added to a microfuge tube:

| | |
|---|---|
| 4 layer DNA dendrimer with ligated Cap03 sequence (50 ng/μL) | 50.0 μL |
| gold nanoparticles previously bound with c(+) oligo (500 ng/μL) | 2.6 μL |
| gold nanoparticles previously bound with a(+) oligo (500 ng/μL) | 2.6 μL |
| 5M NaCl | 4.0 μL |
| 2,4,8 trimethyl psoralen saturated in ethanol | 7.0 μL |

The above reactants are added together, mixed well, placed into a container of water at 65° C. and slow cooled to 42° C. Exposure to UV light (320-400 nm) for 10 minutes (×2) initiates a cross-linking event covalently binding the AuNP oligos to the arms of the DNA dendrimer. Non-cross-linked oligonucleotides are removed via the use of a size exclusion spin column. Small quantities of fluorescent c(+) and/or a(+) oligos are added to some preparations to provide fluorescent labels to assist in tracking dendrimers binding to cellular surfaces.

The specific volumes shown above are for the synthesis of a dendrimer containing approximately 300 nanoparticles per dendrimer. Variation of the volume of the c(+) and a(+) oligonucleotides can be used to vary the number of nanoparticles per dendrimer)

Targeting antibodies were bound to DNA dendrimers by first covalently conjugating a DNA oligonucleotide to either a complete antibody or an antibody fragment (Fab or Fab′(2)) using standard cross-linking condensation conjugation chemistry, followed by hybridizing the antibody-bound oligonucleotide to a complementary sequence on the arms of the dendrimer. This hybridization comprised 31 base pairs with a melting temperature of greater than 65° C., thereby providing a stable complex of dendrimer bound with antibody at physiological temperatures and conditions.

The following components were added to a microfuge tube:

| | |
|---|---|
| 4 layer DNA dendrimer with ligated Cap03 sequence and | 50.0 μL |
| gold nanoparticles (50 ng/μL) | |
| 50% ethelyene glycol in PBS or equivalent | 125.0 μL |
| (e.g. Superfreeze, Pierce Fine Chemicals) | |
| 1X Phosphate Buffered Saline (PBS) | 57.0 μL |
| 5M NaCl | 4.3 μL |
| Antibody (anti-human HLA Class I Mab) with anti-Cap03 | 13.7 μL |
| oligo) previously covalently bound (10 ng/μL as oligo)) | |

The above reactants are combined, gently mixed and incubated at 37° C. for 30 minutes. This formulation is stable at 4° C. for at least six months.

### Example 4

### Use of the Modified Dendrimers for Thermal Ablation of Cancer Cells Grown as Cell Cultures In-Vitro

Cancer test cells were grown in culture, typically in 96 well flat bottom polystyrene plates containing growth media of choice. In one example, Hep G2 cells were plated at 4-8000 cells per well in 100 μL of RPMI 1640 containing 10% FBS, Hepes buffer, 25 mM L-glutamine and gentamicin sulfate (10 mg/L). Growth was allowed to occur over 2-3 days until cells are confluent (15-20,000 cells per well).

DNA dendrimers were manufactured as above to contain a range of gold nanoparticles per dendrimer (<6 to >900 particles, depending on the specific manufacturing conditions (see manufacturing procedure above). DNA dendrimers were added to the cultured Hep G2 cells in the microtiter plate wells to a final concentration of 0.5-10 ng/μL as dendrimer mass. The cells and dendrimers were incubated for 15-180 minutes to allow for binding of the dendrimers to the cell surfaces at 37° C. Binding of dendrimers containing fluorescent labels to cellular surfaces was confirmed using standard fluorescent microscopy.

Dendrimer bound cultured Hep G2 cells are to exposed to RF field as generated by a variable power RF field generator producing radio waves at 13.56 MHz, ranging in power from 0 to 2000 watts. The transmission head (focused end-fired antenna circuit) is held approximately 2-3 cm from the live cells, and RF field exposures of 0 to 5 minutes are performed. Cell death is monitored via the use of standard dye exclusion methods, including the use of MTT reagents (yellow MTT (3-(4,5-Dimethylthiazol-2-yl)-2,5-diphenyltetrazolium bromide, a tetrazole is reduced to purple formazan in the mitochondria of living cells). The absorbance of this colored solution can be quantified by measuring at a certain wavelength by a spectrophotometer. This reduction takes place only when mitochondrial reductase enzymes are active, and therefore conversion can be directly related to the number of viable (living) cells. Successful significant thermal ablation in the presence of DNA dendrimers containing gold nanoparticles is recognized by excess death of cells over control wells containing cells and dendrimers without gold nanoparticles.

### Example 5

### Use of the Modified Dendrimers for Thermal Ablation of Cancer Cells In-Vivo

The modified dendrimers can be used for thermal ablation of cancer cells in-vivo using the methods and devices described in, e.g., Cardinal et al., 2008, Surgery 144:125-132 and Gannon et al., 2008, J. Nanobiotechnology 6:2, each of which is incorporated by reference in its entirety.

What is claimed is:

1. A DNA dendrimer linked to at least one radiation absorbing nanoparticle and at least one targeting moiety.

2. The DNA dendrimer of claim 1, wherein the at least one radiation absorbing nanoparticle is a carbon-based nanoparticle or a metallic nanoparticle.

3. The DNA dendrimer of claim 2, wherein the at least one radiation absorbing nanoparticle is a gold nanoparticle.

4. The DNA dendrimer of claim 1, wherein the radiation absorbing nanoparticle is a nanosphere, a nanorod, a nanoshell, a nanocage, a nanotube or a surface-enhanced Raman scattering nanoparticle.

5. The DNA dendrimer of claim 1 which comprises a capture oligonucleotide associated with an arm of the DNA dendrimer and either or both of the at least one radiation absorbing nanoparticle and the at least one targeting moiety is linked to the DNA dendrimer by hybridization of a carrier oligonucleotide to the capture oligonucleotide.

6. The DNA dendrimer of claim 5, wherein the capture oligonucleotide is linked to a terminus of an extension oligonucleotide and the extension oligonucleotide is hybridized to the arm of the DNA dendrimer.

7. The DNA dendrimer of claim 1, wherein the radiation absorbing nanoparticle is linked to the DNA dendrimer by biotinistreptavidin.

8. The DNA dendrimer of claim 1 which further comprises a tracking label linked to an arm of the DNA dendrimer.

9. The DNA dendrimer of claim 1, wherein the targeting moiety is a protein, a peptide, an aptamer, an antibody, an antibody fragment or a receptor ligand.

10. The DNA dendrimer of claim 1, wherein the dendrimer comprises crosslinked monomers.

11. A method of thermally ablating cells or tissues comprising:
a) contacting the cells or tissues with a DNA dendrimer according to claim 1 such that the targeting moiety binds to a complementary target on the cells or tissues; and
b) exposing the cells or tissues with the bound DNA dendrimer to externally applied electromagnetic radiation at a power and for a time sufficient to cause nanoparticles linked to the DNA dendrimer to emit heat, thereby resulting in thermal ablation of cells or tissues bound to the DNA dendrimer.

12. The method of claim 11, wherein the cells or tissues are contacted with the DNA dendrimer in vivo or ex vivo.

13. The method of claim 11, wherein the cells or tissues are selected from the group consisting of solid tumors, circulating tumor cells, cancer metastases, microorganisms and biological materials for transplantation.

14. The method of claim 11 wherein components of the DNA dendrimer are administered separately and allowed to assemble post-administration on the cells or tissues.

15. A pharmaceutical composition comprising a thermal ablation DNA dendrimer and a pharmaceutically acceptable carrier or excipient, wherein the thermal ablation DNA dendrimer comprises at least one radiation absorbing nanoparticle and at least one targeting moiety.

16. The pharmaceutical composition of claim 15 which comprises a physiologically compatible aqueous buffer.

17. The pharmaceutical composition of claim 15 which is formulated for parenteral administration.

18. A method of making a thermal ablation DNA dendrimer which comprises linking at least one targeting moiety and at least one radiation absorbing nanoparticle to an arm of the DNA dendrimer.

19. The method of claim 18, wherein either or both of the at least one targeting moiety and the at least one radiation absorbing nanoparticle is linked to the arm of the DNA dendrimer by hybridization of a carrier oligonucleotide to a capture oligonucleotide at the terminus of the arm.

20. The method of claim 19 further comprising an extension oligonucleotide hybridized to the arm of the DNA dendrimer and having the capture oligonucleotide at a terminus thereof.

21. The method of claim 19 further comprising a tracking label linked to the arm of the DNA dendrimer.

22. A method for imaging cells or tissues comprising:
a) contacting the cells or tissues with a DNA dendrimer according to claim 1 such that the targeting moiety binds to a complementary target on the cells or tissues, wherein the DNA dendrimer comprises at least one metallic radiation absorbing nanoparticle; and
b) imaging the cells or tissues using the metallic radiation absorbing nanoparticle bound to the cells or tissues.

23. The method of claim 22 wherein components of the DNA dendrimer are administered separately and allowed to assemble post-administration on the cells or tissues.

24. The method of claim 22 wherein the cells or tissues are contacted in vivo.
